(19) United States
(12) Patent Application Publication
(10) Pub. No.: US 2008/0214822 A1
(43) Pub. Date: Sep. 4, 2008

(54) Process For the Preparation of a Leukotriene Antagonist

(75) Inventors: Iolanda Chamorro Gutierrez, Santa Coloma de Farners (ES); Jordi Bosch I Llado, La Girona (ES); Elies Molins I Grau, La Sant Feliu de Llobregat (ES)

Correspondence Address: SCULLY SCOTT MURPHY & PRESSER, PC, 400 GARDEN CITY PLAZA, SUITE 300, GARDEN CITY, NY, 11530, US

(73) Assignee: MEDICHEM, S.A., Sant Joan Despi-Barcelona (ES)

(21) Appl. No.: 11/791,896

(22) PCT Filed: Nov. 30, 2004
(86) PCT No.: PCT/EP04/13598

§ 371 Date: Aug. 24, 2007

(51) Int. Cl. C07D 215/00

(52) U.S. Cl. 546/152

(57) ABSTRACT

The present invention relates to a novel process for the preparation of montelukast sodium, a compound of Formula (1b) and precursors thereof. The invention further concerns the free acid of this compound in crystalline form, obtainable for the first time by the new process.

### FIELD OF THE INVENTION

The present invention relates to a new process for the preparation of a leukotriene antagonist. The invention further relates to 1-[[[(1R)-1-[3-[(1E)-2-(7-chloroquinoline-2-yl)ethenyl]phenyl]-3-[2-(1-hydroxy-1-methylethyl)phenyl]-propyl]thio]methyl]cyclopropaneacetic acid, obtained in solid form for the first time by the described process.

### BACKGROUND OF THE ART

Leukotrienes constitute a group of hormones acting at a local level, which are produced in the living system from arachidonic acid. The most abundant leukotrienes are Leukotriene BA (abbreviated as LTB₄), LTC₄, LTD₄ and LTE₄. The leukotriene biosynthesis begins with the action of the enzyme 5-lipooxygenase on arachidonic acid, giving rise to the epoxide, Leukotriene A₄ (LTA₄), which is converted to other leukotrienes via subsequent enzymatic transformations. Further information on the biosynthesis, metabolism, effect of leukotrienes on living systems and involvement in several illnesses can be found in the book Leukotrienes and Lipoxygenases, ed. J. Rokach, Elsevier, Amsterdam (1989).

Montelukast sodium, a leukotriene antagonist, is useful as anti-asthmatic, anti-allergic anti-inflammatory and cytoprotective agent. Montelukast sodium, chemically known as 1-[[[(1R)-1-[3-[(1E)-2-(7-chloroquinoline-2-yl)ethenyl]phenyl]-3-[2-(1-hydroxy-1-methylethyl)phenyl]-propyl]thio]methyl]cyclopropaneacetic acid monosodium salt, is represented by the following formula (1b):

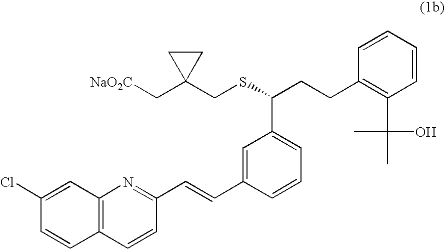

Montelukast sodium and its preparation process was first described in EP 480 717 A1. The disclosed process proceeds via the corresponding methyl ester (5), see Scheme 1. This methyl ester is prepared from the mesylate (3a), which is reacted with methyl 1-(mercaptomethyl)cyclopropaneacetate (4a), generated in situ from methyl 1-(acetylthiomethyl)-cyclopropaneacetate with hydrazine. The methyl ester (5) is subsequently hydrolysed and the resulting hydrolysed product transformed into montelukast sodium (1b). No data about physical properties of the hydrolysed product are given, and the resulting montelukast sodium could only be characterized by chemical analysis and mass spectrometry.

This process is not suitable for the production of montelukast sodium on a large scale. First of all it demands an inconvenient chromatographic purification of the intermediate methyl ester and/or of montelukast. Secondly, the overall yield of montelukast sodium is low which is undesirable under economic considerations.

An alternative preparation method is described in EP 737 186 A1. Therein the dilithium salt of 1-(mercaptomethyl)-cyclopropaneacetic acid is reacted with the mesylate (3). After working-up of the reaction mixture, an organic solution of montelukast is obtained which is then transformed to the dicyclohexylammonium salt of montelukast. Again it is apparent that montelukast cannot be obtained in solid form, and no data about its physical properties are provided.

Still, the process for the preparation of (1b), described in this patent application, is not suitable for operations on a large scale due to the need to employ the reagent n-butyllithium, dissolved in a mixture of hexanes, for the preparation of the dilithium salt. n-Butyllithium is expensive and its handling is delicate and dangerous. It must be used in the absence of any trace of common reactive substances such as water, alcohols, and even atmospheric oxygen, because it is destroyed rapidly and violently on contact with them. Furthermore, the hydrocarbons used as solvent for n-butyllithium, are very volatile and highly flammable.

Additionally, to be used in therapeutic therapy montelukast sodium must be provided in high purity. According to EP 737 186 A1 montelukast sodium is purified by reacting a solution of montelukast with dicyclohexylamine to form the montelukast dicyclohexylammonium salt. This salt is barely soluble in organic solvents and therefore soluble impurities can be removed by filtration. Nevertheless, dicyclohexylamine and hexane are needed for the formation of the dicyclohexylamine salt. Dicyclohexylamine, like hexane, is a substance with high environmental toxicity, particularly to aquatic organisms, it is harmful if swallowed, and hence traces may not remain in the final product.

Furthermore, the dicyclohexylamine salt must subsequently be treated with an acid, the product thus obtained be treated with a sodium ion source and resulting montelukast sodium be isolated. Hence, the preparation of the dicyclohexylamine salt results in an increase in the cost and in the time involved in the manufacturing operations.

Consequently, there exists the need for an efficient preparation method of montelukast sodium, suitable for large scale operations, which provides this product in high yield and with suitable quality.

### SUMMARY OF THE INVENTION

In view of the above exigency for an easy and economically efficient preparation method of montelukast sodium and precursors thereof the present inventors have carried out intensive studies and have accomplished the process of the present invention. This process involves isolation and purification of the montelukast in its acidic form, which compound can be directly transformed to montelukast sodium without the use of n-butyllithium or dicyclohexylamine. Hence it may be used without the extreme safety conditions required in the above mentioned processes. Further the products are obtained with high purity and accordingly no chromatographic purification of the resulting product is necessary. Hence this process allows for the production of montelukast on large scale in an economic manner.

### BRIEF DESCRIPTION OF THE FIGURES

FIG. 1 shows the X-ray powder diffraction pattern of the compound obtained in step c) of Example 1.

FIG. 2 shows the DSC (vented pan) of the compound obtained in step c) of Example 1.

FIG. 3 shows the X-ray powder diffraction pattern of the compound obtained in step c) of Example 2.

FIG. 4 shows the DSC (vented pan) of the compound obtained in step c) of Example 2.

### DETAILED DESCRIPTION OF THE INVENTION

According to the present invention there is provided a process for the preparation of a compound of Formula (1):

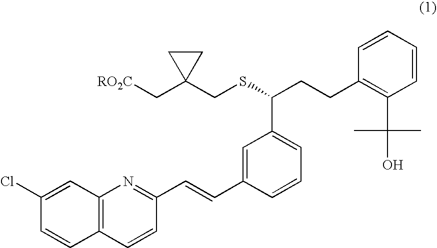

wherein R represents H or Na, which process comprises:
(a) reacting a compound of formula (3):

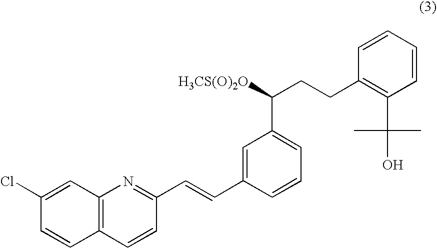

with 1-(mercaptomethyl)-cyclopropane-acetic acid (4) in the presence of a base:

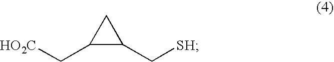

(b) acidifying the obtained solution to yield montelukast, a compound of formula (1a):

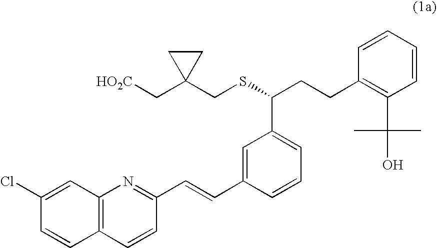

(c) optionally transforming the compound obtained in step (b) into a compound of formula (1b):

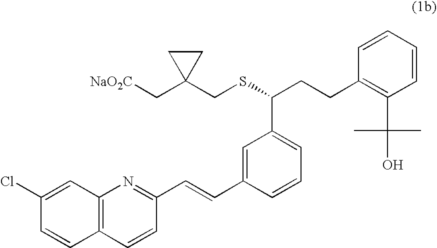

This process is schematically described in Scheme 2.

In this process it is preferred that compound (3) is prepared by reaction of 2-[2-[3(S)-[3-[(1E)-2-(7-chloroquinoline-2-yl)ethenyl]phenyl]-3-hydroxypropyl]phenyl]-2-propanol (2) with a mesylating agent preferably in the presence of a base. Methanesulfonyl chloride or methanesulfonyl anhydride are preferentially employed as mesylating agents. An amine, such as ethyldiisopropylamine, is preferentially used as base. This reaction will generally be carried out in an organic solvent, preferably in an aprotic solvent, more preferably in tetrahydrofurane.

In step (a) of the reaction the base may be an alkali hydroxide, an alkaline earth hydroxide or ammonium, preferably an alkali hydroxide, more preferably lithium hydroxide, sodium hydroxide and potassium hydroxide, and most preferably sodium hydroxide. Without being limited thereto, it is speculated that in this reaction step the dianion of 1-(mercaptomethyl)-cyclopropane-acetic acid (4) is generated in situ. This dianion may then preferably react via its sulfur anion with the mesylate (3) inverting the configuration of the asymmetric C-atom.

This reaction (a) step may be carried out in an organic solvent, preferably in a dipolar aprotic solvent, more preferably in N,N-dimethylformamide (DMF).

It is particularly beneficial when 1-(mercaptomethyl)-cyclopropane-acetic acid (4) is reacted with an alkali hydroxide, an alkaline earth hydroxide or ammonium, as these bases are cheap, may be easily manipulated and represent non flammable materials, in contrast to n-butyllithium in hexane, as used in the EP 737 186 A1, which is an expensive material that is difficult to manipulate and highly flammable.

The acidification step (b) can be carried out in an aqueous medium resulting in the precipitation of montelukast (1a) that can be separated by filtration. Alternatively, in the aqueous medium there may also be present a non-water miscible organic solvent, that can be separated from the water upon acidification, resulting in an organic solvent solution of montelukast (1a) which contains residuals amounts of water. Upon drying this solution for example by distillation, montelukast (1a) is precipitated and can be separated by filtration. The montelukast (1a) obtained in either way is of high purity. A preferred acid is represented by tartaric acid.

Optionally, step (b) may include an additional purification step. This purification may be carried out by digestion of the obtained montelukast (1a) in an organic solvent, preferably in an organic solvent in which montelukast is essentially insoluble such as isopropyl acetate, isopropanol, ethyl acetate or acetonitrile.

The optional transformation step (c) of the montelukast (1a) in montelukast sodium (1b) is preferably carried out by mixing the montelukast (1a) either as a solid or dissolved in an alcohol, such as ethanol with an aqueous solution of one equivalent of sodium hydroxide, followed by evaporation or lyophilization of the solvent.

The process of the present invention not only allows the preparation of montelukast sodium (1b) with a therapeutically acceptable purity, but also employs operations which can be easily scaled up.

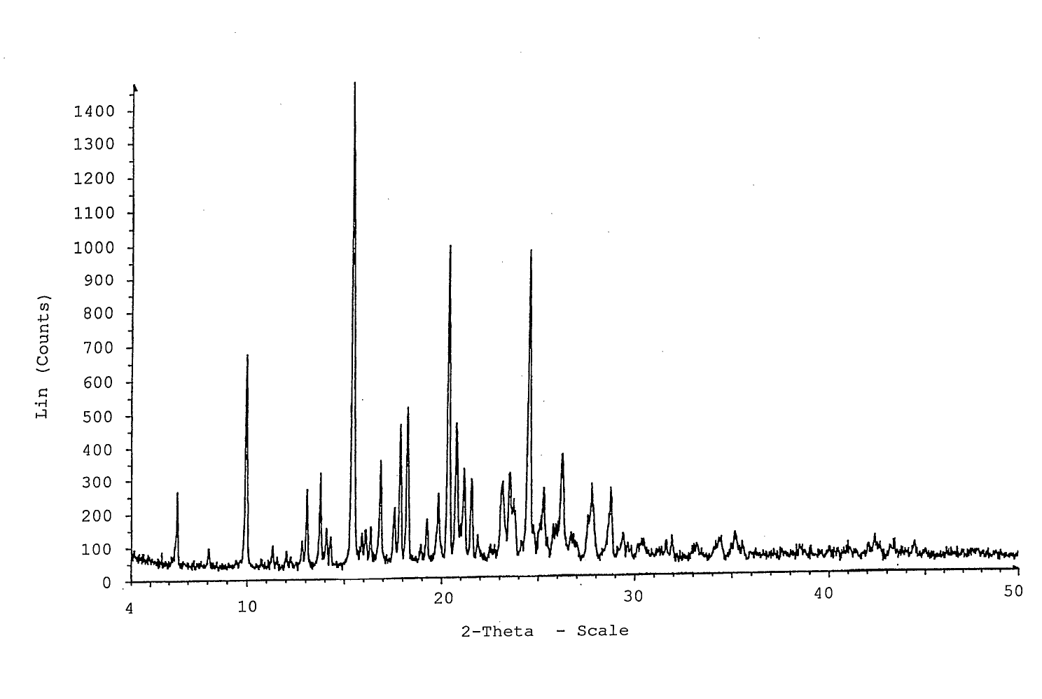

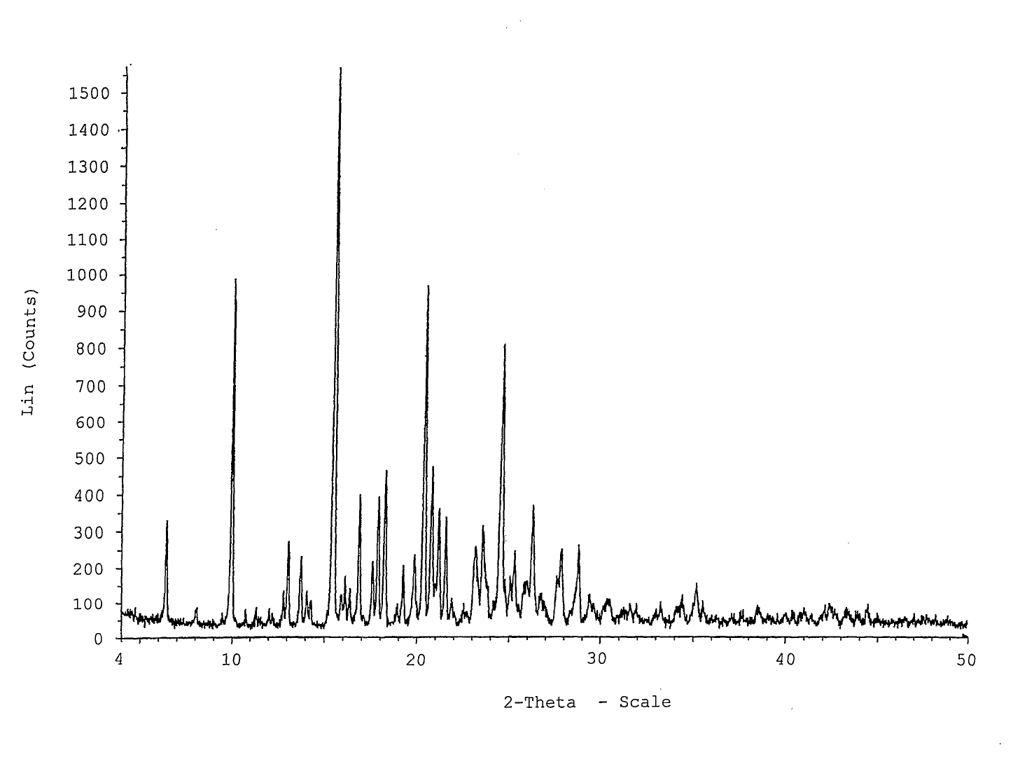

Additionally, this process allows for the first time the preparation of montelukast (1a) in crystalline form. Furthermore it was possible for the first time to obtain an X-ray powder diffraction pattern of montelukast, cf. FIG. 1 or FIG. 3. Hence montelukast obtainable by the present process represents embodiments of the present invention according to claims 10 to 14.

### EXAMPLES

In the following examples of the process according to the present invention are given. It will be apparent that these examples are given for illustrative purposes only and are not intended to limit the scope of the invention.

### Example 1

### Preparation of Montelukast Sodium (1b) from Compounds (4) and (2)

### Step a) Preparation of 2-[2-[3(S)-[3-[(1E)-2-(7-chloroquinoline-2-yl)ethenyl]phenyl]-3-methanesulfonyloxypropyl]phenyl]-2-propanol (3)

Ethyldiisopropylamine (22.55 mL) is added to a stirred solution of 2-[2-[3(S)-[3-(1E)-2-(7-chloroquinoline-2-yl)ethenyl]phenyl]-3-hydroxypropyl]phenyl]-2-propanol (2) (51.12 g, 97.8% purity, 109 mmol) in tetrahydrofurane (100 mL) in a 1000 mL flask, kept at room temperature under a nitrogen atmosphere. The resulting brown solution is cooled to −22.5±2.5° C. with an acetone-dry ice bath. Methanesulfonyl chloride (9.8 mL) is slowly added during 15 min by means of an addition funnel while the temperature of the solution is kept at −22.5±2.5° C. during all the addition. The resulting viscous dark brown solution was kept at −22.5±2.5° C. for an additional hour. Acetonitrile (300 mL) was slowly added over one hour and 50 min while the temperature was kept at −22.5±2.5° C., resulting in the precipitation of a solid. The resulting suspension was kept at −22.5±2.5° C. over 2 hours, and the mixture was filtered under nitrogen. The collected solid was washed with a small amount of cold acetonitrile and dried under vacuum while it was contained in a flask kept over a nitrogen-acetone bath, which was always kept bellow −10° C., resulting in the isolation of mesylate (3) (49.97 g, 85.4% yield).

### Step b) Preparation of 1-[[(1R)-1-[3-[(1E)-2-(7-chloroquinoline-2-yl)ethenyl]phenyl]-3-[2-(1-hydroxy-1-methylethyl)phenyl]propyl]thio]methyl]-cyclopropaneacetic acid (1a)

A brown-orange mixture of mesylate (3) obtained in step a) above (20 g, 37.3 mmol), 1-(mercaptomethyl)-cyclopropaneacetic acid (4) (8.18 g, 56 mmol), sodium hydroxide (4.48 g, 112 mmol) and dimethylformamide (DMF) (129 mL) was stirred in a 500 mL flask under nitrogen on an ice-salt bath over 6 hours while the temperature was kept between −5 and 0° C. Water (60 mL), isopropyl acetate (120 mL) and a solution of 8.9 g of sodium chloride in 60 mL of water were sequentially added. The addition of water caused a small exothermic reaction. The resulting mixture was stirred for 15 min and both phases were separated. Water (120 mL) was added to the organic phase and the resulting mixture was stirred for 15 min before both phases were separated. The aqueous phase, containing the product in the form of its sodium salt, was acidified with a 0.5 M aqueous solution of tartaric acid till a pH of 4 to 5 was reached, resulting in the precipitation of acid (1a). The mixture was filtered and the solid was washed with water and dried in vacuo at 40° C., yielding acid (1a) (15.55 g, 71.1% yield, 95% purity by HPLC).

### Step c) Purification of 1-[[[(1R)-1-(3-[(1E)-2-(7-chloroquinolin-2-yl)ethenyl]phenyl]-3-[2-(1-hydroxy-1-methylethyl)phenyl]propyl]thio]methyl]-cyclopropaneacetic acid (1a) by treatment with isopropyl acetate

Isopropyl acetate (5 mL) was added to 1 g of the product obtained in the previous step. The resulting suspension was refluxed for 10 min and then kept at 20±5° C. for 1 hour. The mixture was filtered and the obtained solid was washed with isopropyl acetate and dried in vacuo at 40° C. (0.856 g, 85.6% yield, 97.9% purity by HPLC).

The X-ray diffractogram was registered using a RX SIEMENS D5000 diffractometer with a vertical goniometer and a copper anodic tube, radiation CuK_{α}, λ=1.54056 Å.

The X-ray diffraction diagram is shown in FIG. 1.

Peak characteristic positions expressed in d-spacings (Å) are: 13.77, 10.99, 8.85, 8.22, 7.80, 7.35, 6.89, 6.77, 6.42, 6.28, 6.18, 5.72, 5.56, 5.49, 5.41, 5.24, 5.03, 4.95, 4.85, 4.68, 4.60, 4.46, 4.35, 4.27, 4.18, 4.11, 4.05, 3.93, 3.83, 3.77, 3.62, 3.54, 3.51, 3.42, 3.38, 3.29, 3.20, 3.09, 3.03, 3.01, 2.93, 2.85, 2.82, 2.80, 2.70, 2.62, 2.60, 2.54, 2.52

Melting Point: 152.2-153.4° C.

Optical Rotation: +99.42° (c=1%; D; Methanol; T^{a}=20° C.)

DSC measurements were carried out in vented pan at a scan rate of 10° C./minute from 25.0° C. to 180.0° C. under a nitrogen purge with a Pyris I DSC available from METTLER-TOLEDO.

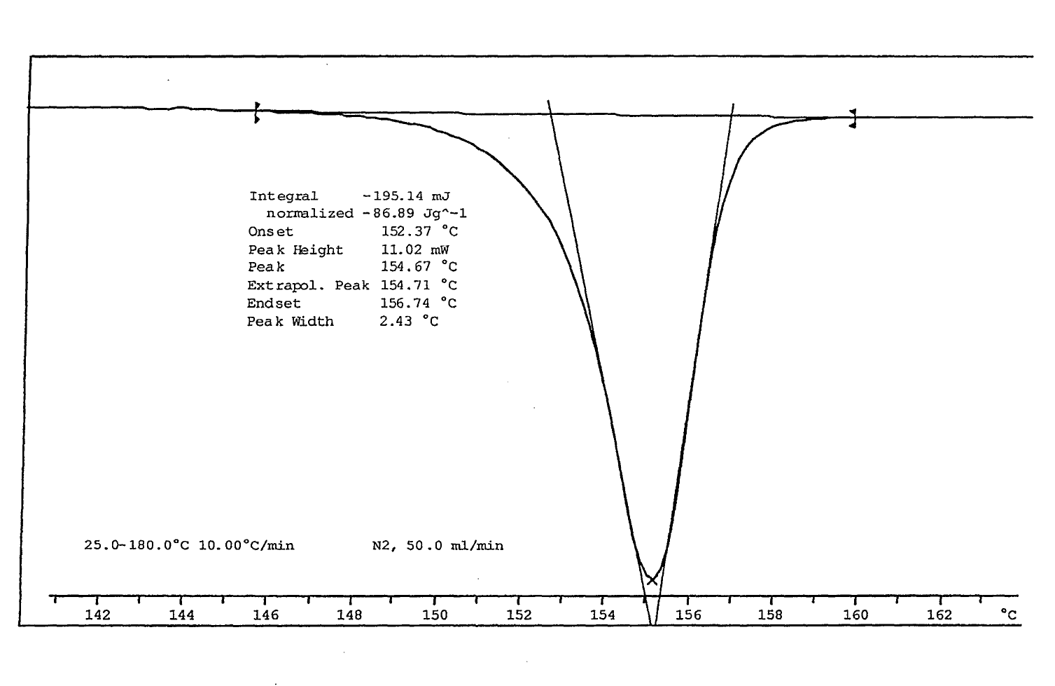

The DSC of the product possesses the characteristic endothermic point at 154.67° C. with a temperature onset of 152.37° C. (see FIG. 2).

### Step d) Preparation of sodium 1-[[[(1R)-1-[3-[(1E)-2-(7-chloroquinoline-2-yl)ethenyl]phenyl]-3-[2-(1-hydroxy-1-methylethyl)phenyl]propyl]thio]methyl]cyclopropaneacetate (1b)

One equivalent of 1N NaOH is added to an ethanolic solution of montelukast (1a), obtained in the previous step c). The solvent is evaporated and water is added to the resulting residue till a solution is obtained. The resulting solution is concentrated to dryness with a rotatory evaporator at 50° C., resulting in the isolation of compound (1b).

### Example 2

### Preparation of Montelukast from Compounds (4) and (2)

### Step a) Preparation of 2-[2-[3(S)-[3-[(1E)-2-(7-chloroquinoline-2-yl)ethenyl]phenyl]-3-metanesulfoyloxipropyl]phenyl]-2-propanol (3)

Ethyldiisopropylamine (45.09 mL) was added to a stirred solution of 2-[2-[3(S)-[3-[(1E)-2-(7-chloroquinoline-2-yl)ethenyl]phenyl]-3-hydroxypropyl]phenyl]-2-propanol (2) (100.42 g, 99.58% purity, 218 mmol) in tetrahydrofurane (200 mL), kept at room temperature under nitrogen. The resulting brown solution was cooled at −22.5±2.5° C. with an acetone-dry ice bath. Methanesulfonyl chloride (19.65 mL) was slowly added over 15 min using an addition funnel, while the temperature of the solution was kept at −22.5±2.5° C. The resulting viscous dark brown solution was kept at this temperature for an additional hour. Acetonitrile (600 mL) was slowly added over 1 hour and 25 min while the temperature was kept at −22.5±2.5° C., resulting in the precipitation of a solid. The resulting suspension was kept at −22.5±2.5° C. for 2 additional hours and the mixture was filtered under nitrogen. The solid was washed with a small amount of cold acetonitrile and dried in vacuo while it was contained in a flask cooled in a nitrogen-acetone bath, which was always kept bellow −10° C., resulting in the isolation of the mesylate (3) (74.61 g, 63.7% yield).

### Step b) Preparation of 1-[[[(1R)-1-[3-[(1E)-2-(7-chloroquinoline-2-yl)ethenyl]phenyl]-3-[2-(1-hydroxy-1-methylethyl)phenyl]propyl]thio]methyl]-cyclopropaneacetic acid (1a)

The brown-orange suspension obtained by mixing the mesylate (3) obtained in the previous step a) (38 g, 70.9 mmol), 1-(mercaptomethyl)-cyclopropaneacetic acid (4) (15.55 g, 106 mmol), solid sodium hydroxide (8.51 g, 213 mmol) and dimethylformamide (DMF) (228 mL) in a 1000 mL flask kept under nitrogen on an ice-salt bath was stirred for 6 hours at a temperature of between −5 and 0° C. Water (114 mL), isopropyl acetate (228 ml) and a solution of sodium chloride (16.91 g) in 114 mL of water were sequentially added. The initial addition of water produced a small exothermic reaction. The resulting mixture was stirred for 25 min and both phases were separated. Water (228 mL) was added to the organic phase and the resulting mixture was stirred for 20 minutes. Both phases were separated and 150 mL of isopropyl acetate were added to the aqueous phase containing the product in the form of its sodium salt. Tartaric acid was added to the resulting mixture till a pH between 4 and 5 was achieved. Both phases were separated and the organic phase was treated with active charcoal for 1 hour at room temperature and filtered through a Celite pad. The resulting solution was concentrated in vacuo to about 80% of the initial volume and stirred overnight at room temperature. The resulting suspension, containing montelukast (1a) was filtered and the solid was washed with isopropyl acetate. The resulting solid was dried in vacuo at 40° C. yielding montelukast (1a) (21.31 g, 51.3% yield, 96.85% purity by HPLC).

### Step c) Purification of 1-[[[(1R)-1-[3-[(1E)-2-(7-chloroquinoline-2-yl)ethenyl]phenyl]-3-(2-[1-hydroxy-1-methylethyl)phenyl]propyl]thio]methyl]-cyclopropaneacetic acid (1a) by treatment with isopropyl acetate

Isopropyl acetate (40 mL) was added to 20 g of the product obtained in the previous step. The resulting suspension was refluxed for 10 min and then kept at 20±5° C. for 1 hour. The mixture was filtered and the solid was washed with isopropyl acetate and dried in vacuo at 40° C. (16.59 g, 82.95% yield, 98.10% purity by HPLC).

This purification procedure employing isopropyl acetate was repeated starting with 8.5 g obtained in the previous step, resulting in the isolation of 8.25 g of purified product (97.1% yield, 98.44% of purity by HPLC).

The X-ray diffractogram was registered using a RX SIEMENS D5000 diffractometer with a vertical goniometer and a copper anodic tube, radiation CuK_{α}, λ=λ1.54056 Å.

X-ray diffraction diagram as shown in FIG. 3.

Peak characteristic positions expressed in d-spacings (A) are: 13.77, 10.99, 8.85, 8.22, 7.80, 7.35, 6.89, 6.77, 6.42, 6.28, 6.18, 5.72, 5.56, 5.49, 5.41, 5.24, 5.03, 4.95, 4.85, 4.68, 4.60, 4.46, 4.35, 4.27, 4.18, 4.11, 4.05, 3.93, 3.83, 3.77, 3.62, 3.54, 3.51, 3.42, 3.38, 3.29, 3.20, 3.09, 3.03, 3.01, 2.93, 2.85, 2.82, 2.80, 2.70, 2.62, 2.60, 2.54, 2.52

Combustion Analysis: C=71.67%; H=6.28%; N=2.24%; S=5.26%; Cl=6.19%

Expected: C=71.71%; H=6.19%; N=2.39%; S=5.47%; Cl=6.05%

Melting Point: 152.4-153.3° C.

Optical Rotation: +98.98° (c=1%; D; Methanol; T^{a}=20° C.)

DSC measurements were carried out in vented pan at scan rate of 10° C./minute from 25.0° C. to 180.0° C. under a nitrogen purge with a Pyris I DSC available from METTLER-TOLEDO.

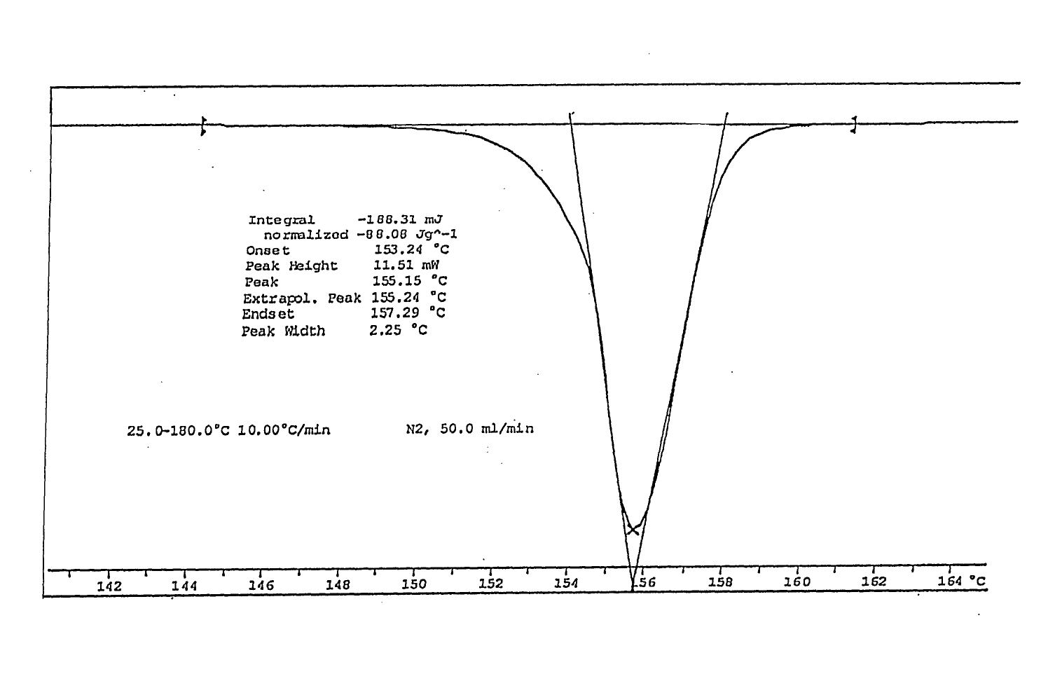

The DSC of the product possesses the characteristic endothermic point at 155.15° C. with a temperature onset of 153.24° C. (see FIG. 4).

### Step d) Preparation of sodium 1-[[[(1R)-1-[3-[(1E)-2-(7-chloroquinoline-2-yl)ethenyl]phenyl]-3-[2-(1-hydroxy-1-methylethyl)phenyl]propyl]thio]methyl]cyclopropaneacetate (1b)

Montelukast (1a) obtained in the previous step was added on an aqueous solution containing 1 equivalent of NaOH. The resulting mixture was stirred at room temperature till a solution was obtained. The resulting solution was filtered and dried at the rotatory evaporator at 50° C., resulting in the isolation of montelukast sodium (1b) (92.8% yield, 98.78% purity by HPLC, Titration=100.29%, Water content (Karl Fischer (K.F.))=2.90%).

### Example 3

### Preparation of Mesylate (3) by Mesylation of Alcohol (2) with Methanesulfonic Anhydride

Ethyldiisopropylamine (1.13 mL) plus tetrahydrofurane (0.5 mL) was added to a stirred solution of 2-[2-[3(S)-[3-[(1E)-2-(7-chloroquinoline-2-yl)ethenyl]phenyl]-3-hydroxypropyl]phenyl]-2-propanol (2) (2.0 g, 97.8% purity, 4.4 mmol) in tetrahydrofurane (2.5 mL) which was kept under nitrogen at room temperature in a 100 mL flask. The resulting brown solution was cooled to −22.5±2.5° C. over an acetone-dry ice bath. A solution of methanesulfonic anhydride (1.14 g) in tetrahydrofurane (1.5 mL) was slowly added via an addition funnel over 3 min to the stirred solution kept at −22.5±2.5° C. An additional quantity of 1 mL of tetrahydrofurane was added after 5 min to the viscous solution in order to allow stirring. After 2.5 hours of stirring of the resulting viscous dark brown solution at −22.5±2.5° C., it was cooled down to −35±5° C. and acetonitrile (12 mL) was slowly added over 15 min while the temperature was kept at −35±5° C. This resulted in the precipitation of a solid that was filtered under nitrogen after keeping the suspension at −35±5° C. for 1.5 hours. The filtered solid was washed with a small amount of cold acetonitrile and dried in vacuo while it was contained in a flask kept below −10° C. over a nitrogen-acetone bath, resulting in the isolation of mesylate (3) (1.66 g, 70.94% yield).

### Example 4

### Preparation of the Montelukast (1a) from Mesylate (3) and Compound (4)

The brown-orange suspension resulting from the mixture of mesylate (3) (obtained following the same protocol as described above for examples 1 and 2, 37 g, 69 mmol) 1-(mercaptomethyl)-cyclopropaneacetic acid (4) (15.14 g, 103 mmol), solid sodium hydroxide (8.28 g, 207 mmol) and dimethylformamide (DMF) (222 mL) was stirred in a 1 L flask under nitrogen on an ice-salt bath for 6 hours while the temperature was kept between −5 and 0° C.

Water (111 mL), isopropyl acetate (222 mL) and a solution of sodium chloride (16.47 g) in water (111 mL) were sequentially added. A small exothermic reaction was observed after the addition of water. The resulting mixture was stirred for 15 min and both phases were separated. Water (222 mL) was added to the organic phase and the resulting mixture was stirred for 20 min. Both phases were separated and isopropyl acetate (222 mL) was added to the aqueous phase, that contained the product in the form of its sodium salt. The resulting mixture was stirred for 15 min and the aqueous phase was separated, mixed with isopropyl acetate (146.15 mL) and acidified with tartaric acid to a pH between 4 and 5. After stirring for 15 min, both phases were separated and the content of montelukast (1a) present in the organic phase was measured by a potentiometric tritation with perchloric acid. The organic phase was concentrated in vacuo to 2 volumes of solution per weight of acid present in the initial solution, resulting in precipitation of the acid. The resulting suspension was stirred for 2 hours at room temperature and for 2 additional hours on an ice-water bath and then filtrated. The solid was washed with isopropyl acetate and dried in vacuo at 40° C., resulting in the isolation of acid (1a) (17.21 g, 42.54% yield, 98.23% purity HPLC).

### Example 5

### Preparation of Montelukast Sodium (1b) from a Basic Aqueous Solution of the Montelukast (1a)

50.05 g (titration=99.91%, 0.085 mols) of montelukast (1a) (obtained following the same protocol as described in example 4) were added to a basic aqueous solution prepared by mixing 3.927 g (0.097 mols) of sodium hydroxide in 865.00 ml of water. The resulting suspension was stirred until a yellow solution was obtained, which was titrated with tetra n-butyl ammonium hydroxide to check that the salification was complete.

The yellow solution was filtered to remove any particulates impurities, resulting in a clear solution, that was lyophilized in a LYOBETA 25 (cycle: 3.30 h freezing at −45° C. and 17 h primary drying at −10° C., 0.200 mbar)

The process took one day, after which a yellow porous solid

(1b) was obtained (53.70 g, 97.73% yield, 97.38% purity by HPLC, assay 99.85%, water content (KF)=5.76%, lod (loss on drying)=3.47% (80° C., 3 hours), [α]=+91.17, (c=1%; D=Methanol; T^{a}=20° C.)

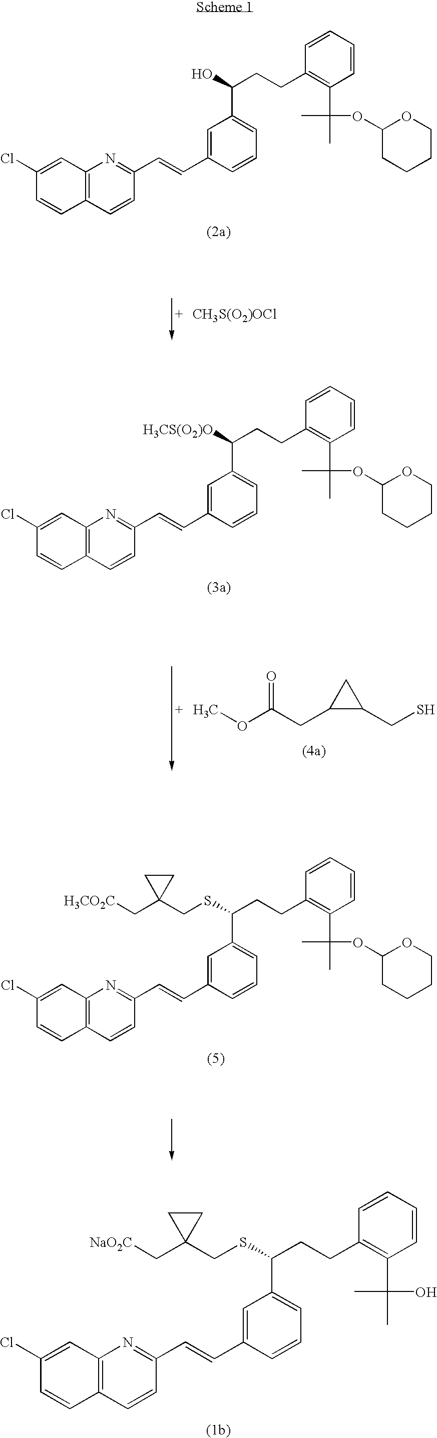

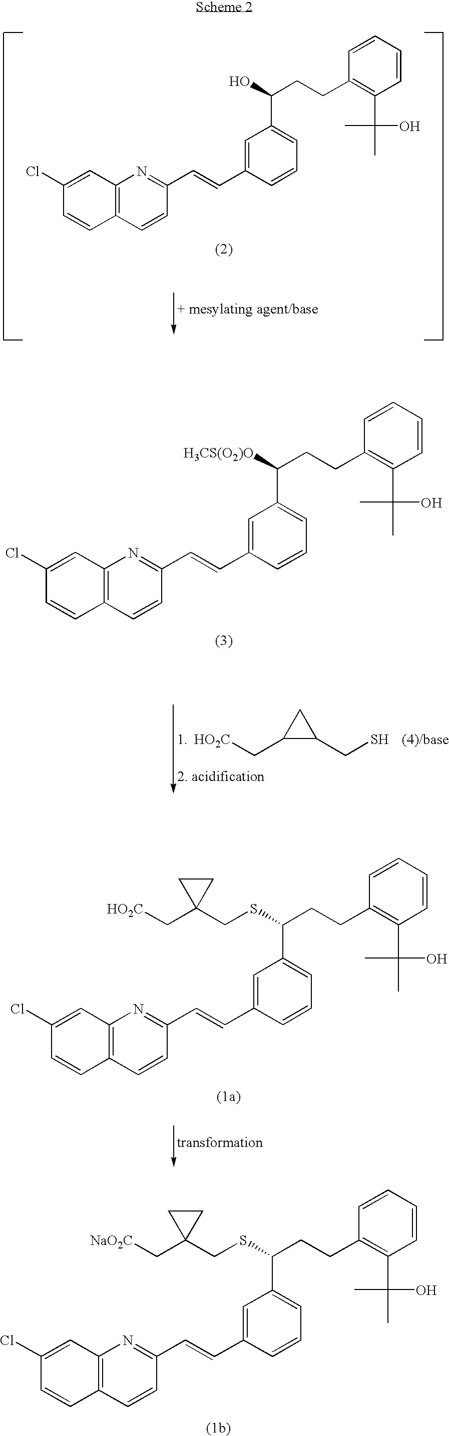

1. A process for the preparation of a compound of Formula (1): 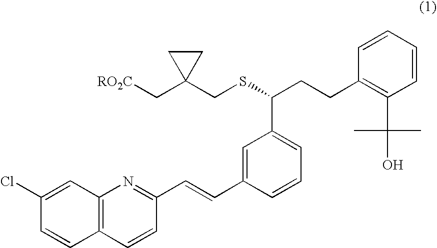
wherein R represents H or Na, which process comprises:
(a) reacting a compound of formula (3): 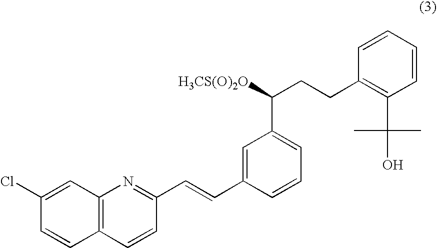
with 1-(mercaptomethyl)-cyclopropane-acetic acid (4) in the presence of a base selected from the group consisting of an alkali hydroxide, an alkaline earth hydroxide and ammonia: 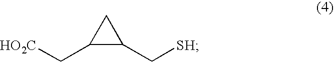
(b) acidifying the obtained solution to yield a compound of formula (1a): 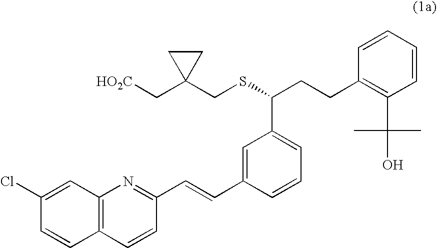
(c) and optionally transforming the compound obtained in step (b) into a compound of formula (1b):

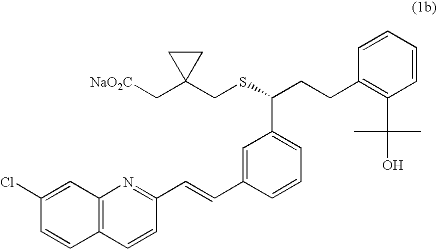

2. The process according to claim 1, wherein the base in step (a) represents an alkali hydroxide.

3. The process according to claim 2, wherein the alkali hydroxide in step (a) is selected from lithium hydroxide, sodium hydroxide and potassium hydroxide.

4. The process according to claim 3, wherein the alkali hydroxide in step (a) is sodium hydroxide.

5. The process according to claim 1, wherein the base in step (a) generates the dianion of 1-(mercaptomethyl)-cyclopropane-acetic acid (4).

6. The process according to claim 5, wherein the base in step (a) generates the dianion of 1-(mercaptomethyl)-cyclopropane-acetic acid (4) in situ.

7. The process according to claim 1, wherein the acidification step (b) is carried out in an aqueous medium.

8. The process according to claim 7, wherein the aqueous medium contains at least one non-water miscible organic solvent.

9. The process according to claim 1, wherein tartaric acid is used in the acidification step (b).

10. The process according to claim 1, wherein the transformation step (c) is carried out by mixing the compound of formula (1 a) as a solid or dissolved in an alcohol, with one equivalent of sodium hydroxide present in an aqueous solution, followed by evaporation or lyophilization of the solvent.

11. The process according to claim 10, wherein the alcohol used in the transformation step (c) is ethanol.

12. The process according to claim 1, wherein the compound of formula (3) is prepared by reacting a compound of formula (2) with a mesylating agent in an organic solvent and in the presence of a base:

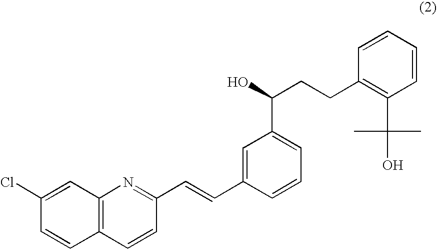

13. The process according to claim 12, wherein the mesylating agent used in the preparation of the compound of formula (3) is methanesulfonyl chloride or methanesulfonyl anhydride.

14. The process according to claim 12, wherein the organic solvent used in the preparation of the compound of formula (3) is an aprotic solvent.

15. The process according to claim 14, wherein the aprotic solvent is tetrahydrofurane.

16. The process according to claim 12, wherein the base used in the preparation of the compound of formula (3) is ethyldiisopropylamine.

17. The process according to claim 1, wherein step (b) includes an additional purification step.

18. The process according to claim 17, wherein the additional purification step is carried out by digestion of the compound of formula (1a) in an organic solvent.

19. The process according to claim 18, wherein the organic solvent used in the additional purification step is an organic solvent in which the compound of formula (1a) is essentially insoluble.

20. The process according to claim 19, wherein the organic solvent in which the compound of formula (1a) is essentially insoluble is selected from isopropyl acetate, isopropanol, ethyl acetate and acetonitrile.
